(19) United States
(12) Patent Application Publication
(10) Pub. No.: US 2024/0245802 A1
(43) Pub. Date: Jul. 25, 2024

(54) PHARMACEUTICAL COMPOSITION FOR PREVENTING OR TREATING BRAIN TUMORS COMPRISING EXTRACELLULAR VESICLES LOADED WITH MITOCHONDRIA-TARGETING PHOTOSENSITIZER AS AN ACTIVE INGREDIENT

(71) Applicant: ExoTheranostics, Inc., Incheon (KR)

(72) Inventor: Min Suk SHIM, Incheon (KR)

(21) Appl. No.: 18/394,973

(22) Filed: Dec. 22, 2023

(30) Foreign Application Priority Data
Jan. 19, 2023 (KR) 10-2023-0008002
Dec. 14, 2023 (KR) 10-2023-0181783

(51) Int. Cl. A61K 47/69; A61K 41/00

(52) U.S. Cl. CPC A61K 47/6901; A61K 41/0071

(57) ABSTRACT

The present invention is related to a brain targeted drug delivery system (DDS). The DDS penetrates the blood brain barrier and delivers the drug by using extracellular vesicles (bEVs) derived from brain vascular endothelial cells. The present invention provides a composition for enhancing PDT efficacy comprising bEV loaded with TPP-Ce6. The bEV of the present invention penetrates the BBB effectively without cytotoxicity, and the bEV loaded with TPP-Ce6 is selectively absorbed by brain tumor cells, and releases TPP-Ce6 under a light condition, and the TPP-Ce6 accumulates preferably in mitochondria first. This invention is expected to enhance therapeutic efficacy of PDT and minimize side effects in the treatment of brain tumors.

### CROSS-REFERENCE TO RELATED APPLICATIONS

The present application claims priority to Korean Patent Application No. 10-2023-0008002, filed on Jan. 19, 2023, and Korean Patent Application No. 10-2023-0181783 filed on Dec. 14, 2023, the entire disclosures of which are incorporated herein by reference.

### TECHNICAL FIELD

The following description relates to brain endothelial cell-derived extracellular vesicle (bEV) as a drug delivery system (DDS) for delivering drugs to the brain, and use of the bEV loaded with mitochondrial targeting TPP-Ce6 for treating brain tumors and enhancing photodynamic therapy efficacy.

### BACKGROUND ART

Glioblastoma (GBM) is the most aggressive malignant brain tumor and for patients with GBM survival rate is low and prognosis is poor. Patients with GBM have a median survival time of less than 1 year, and have a 5-year survival rate of less than 4%. Despite advances in the multimodal treatment of surgical resections with radiotherapy and/or chemotherapy in a clinic, the effectiveness of this treatment remains limited owing to the inherent resistance of glioma cells to chemo- and radiotherapy along with poor transport of therapeutics to target brain tumors.

Recently, clinical trials of photodynamic therapy (PDT) have shown great promise for the eradication of malignant brain tumors. PDT, wherein photosensitizers generate cytotoxic reactive oxygen species (ROS) under light, can selectively kill infiltrating malignant brain tumors with minimal damage to nearby healthy tissues because light can be precisely focused on a specific tumor region. Although PDT can selectively destroy tumors, the therapeutic efficacy of PDT for brain tumors has been limited because for photosensitizers the ability to penetrate the protective blood-brain barrier (BBB) is poor.

With the remarkable recent advances in nanocarrier-based drug delivery, a variety of nanocarriers with surface decoration of BBB-targeting ligands have been developed to penetrate the BBB via receptor-mediated endocytosis and transcytosis. Moreover, nanoparticles can significantly improve the pharmacokinetics and tumor-targeting efficacy of encapsulated drugs. However, despite the promise of nanocarrier-based drug delivery, its translation to clinical settings remains a challenge, mainly due to the inherent toxicity of nanocarriers, particularly artificial ones. Moreover, modifying the surface of nanocarriers with BBB-targeting ligands is often complicated and often requires laborious engineering processes. Therefore, the development of highly biocompatible nanocarriers capable of efficient BBB penetration is indispensable to the task of facilitatingh the translation of nanocarrier-based brain tumor therapy to clinical use.

Extracellular vesicles (EVs), cell-derived nanoscale (30-150 nm) vesicles for intercellular communication, have recently emerged as versatile natural nanocarriers for drug delivery. Due to their endogenous origin, they possess outstanding biocompatibility, long blood circulation half-life, and no or minimal immunogenicity. Moreover, EVs are effective endogenous nanocarriers that can cross the BBB via receptor-mediated endocytosis. The significance of EVs as endogenous nanocarriers is that they express tissue-specific surface proteins derived from parental cells. Therefore, it is hypothesized that EVs derived from brain endothelial cells would display brain endothelial-specific proteins for efficient transport of therapeutic agents across the BBB via interaction with brain endothelial cells.

In recent years, subcellular organelle-specific delivery of therapeutics has shown great promise to enhance therapeutic efficacy while minimizing side effects. Mitochondria are vital subcellular organelles in eukaryotic cells, and they regulate cellular functions because they are energy powerhouses of the cells. Mitochondria are also decisive regulators of apoptosis.

Because mitochondrial damage triggers mitochondrial dysfunction and apoptosis, targeting mitochondria has been recognized as an effective strategy for cancer treatment. In particular, mitochondria are highly susceptible to oxidative damage induced by excessive ROS generation. Thus, using mitochondria-targeted PDT has emerged as a promising strategy for treating cancer efficiently. Many efforts have been made to develop nanocarriers incorporated with mitochondria-targeting moieties for the targeted delivery of photosensitizers to the mitochondria of cancer cells. However, mitochondria-targeted nanocarriers do not guarantee high mitochondrial accumulation of photosensitizers due to the problem that photosensitizers leak prematurely, before they reach the target mitochondria, which inevitably limits the efficacy of PDT.

The present inventors completed the present invention by carefully studying a method for increasing the efficacy of photodynamic therapy by penetrating the BBB and targeting mitochondria in brain tumors thereby efficiently accumulating photosensitizers in mitochondria in brain tumor cells.

### SUMMARY OF THE DISCLOSURE

### Technical Goals

Purposes of the present disclosure are to provide brain endothelial cell-derived extracellular vesicles (bEVs) that can effectively penetrate the BBB as a drug delivery system (DDS) that can deliver drugs to the brain, to provide a composition for enhancing photodynamic therapy efficacy by loading the DDS with a photosensitizer, and to more effectively enhance photodynamic therapy efficacy by bonding a mitochondrial target substance to the photosensitizer.

However, the technical purposes to be achieved by the present disclosure are not limited to the above-mentioned purposes, and other purposes not mentioned will be clearly understood by those skilled in the art from the following description.

### Technical Solutions

In order to achieve the above-mentioned purposes, the present disclosure provides a drug delivery system for penetrating the brain-vascular barrier that contains extracellular vesicles (bEVs) derived from brain vascular endothelial cells as active ingredients.

Further, the present disclosure provides a composition for enhancing the effectiveness of photodynamic therapy, including the brain vascular endothelial cell-derived extracellular vesicles (bEVs) loaded with a photosensitizer as an active ingredient.

Further, the present disclosure provides a composition for treating brain tumors, which includes bEVs loaded with a photosensitizer as an active ingredient.

In an aspect of the present disclosure, the composition may be administered before photodynamic therapy, preferably 3 to 6 hours before photodynamic therapy, and more preferably 4 hours before photodynamic therapy.

In another aspect of the present disclosure, the photosensitizer may be one to which triphenyl phosphonium (TPP) is bonded.

In another aspect of the present disclosure, the photosensitizer may be chlorine e6 (Ce6).

In another aspect of the present disclosure, the brain vascular endothelial cells may be brain vascular endothelial cells derived from humans or mice, for example, hCMEC/D3 or bEnd.3 cells.

In another aspect of the present invention, the brain tumor may be glioblastoma.

Further, the present disclosure provides a method of manufacturing TPP-conjugated Ce6 (TPP-Ce6), including the following steps:
(1) stirring after mixing EDC.HCl, DMAP, and chlorin e6 (Ce6) under dimethylformamide (DMF);
(2) Adding 2-hydroxyethyl triphenylphosphonium to the mixture;
(3) stirring the mixture of step (2) and precipitating it using diethyl ether; and (4) purifying the precipitate by silica gel column chromatography using an elution with a gradient of 80/19/1 dichloromethane/methanol/triethylamine in dichloromethane.

In an aspect of the present disclosure, step (1) may be to mix 100 mg of Ce6 with 128.8 mg of EDC·HCl and 82.1 mg of DMAP under 5 mL DMF and agitate for 2 hours in a dark environment at room temperature.

In another aspect of the present disclosure, step (2) may be to add 2 mL DMF containing 324.3 mg of 2-hydroxyethyl TPP to the mixture of step (1) and stir the mixture at room temperature for 24 hours.

Further, the present disclosure provides a method of manufacturing a TPP-Ce6 loaded bEV by culturing brain vascular endothelial cell-derived extracellular vesicles (bEVs) with TPP-conjugated Ce6 (TPP-Ce6) prepared together by the above-described method.

In addition, the present disclosure provides a bEV loaded with TPP-Ce6 manufactured by the method.

Further, the present disclosure provides a brain tumor treatment method including the following steps:
(A) administering a bEV loaded with TPP-Ce6 to a subject;
(B) performing photodynamic therapy on the subject.

In an aspect of the present disclosure, the subject may be a mammal, particularly a human, that needs treatment for a brain tumor.

In another aspect of the present disclosure, the administration of step (A) may be intravenous administration.

In another aspect of the present disclosure, step (B) may be performed after step (A) has been performed for 4 hours.

### Advantageous Effects

The present disclosure provides extracellular vesicles (bEV) derived from brain vascular endothelial cells capable of effectively penetrating the BBB and delivering drugs to the brain as a drug delivery system, and provides a PDT efficacy enhancing composition comprising a photosensitizer which is loaded on the bEV. The photosensitizer loaded on the bEV effectively penetrates the BBB, is introduced into brain tumor cells, and the photosensitizer could accumulate preferentially in mitochondria of the cells by being conjugated with a mitochondrial targeting molecule. Since the drug delivery system of the present disclosure effectively penetrates the BBB while having very low cytotoxicity, the present disclosure makes it possible to perform photodynamic therapy with improved therapeutic efficacy and minimized side effects.

### BRIEF DESCRIPTION OF DRAWINGS

FIG. 1A is a synthetic scheme of TPP-Ce6.

FIG. 1B is a schematic illustration of the mitochondria-targeted PDT using bEV(TPP-Ce6) that crosses the BBB. After bEV(TPP-Ce6) is intravenously injected into orthotopic GBM-xenografted mice, it penetrates the blood-brain barrier through transferrin receptor-mediated transcytosis, followed by internalization into U87MG brain cancer cells. After endocytosis, TPP-Ce6 is efficiently released from bEV(TPP-Ce6) upon irradiation with 660-nm light. The released TPP-Ce6 preferentially accumulates within mitochondria and generates ROS upon light irradiation. The ROS induces mitochondrial damage and apoptotic cell death.

In FIG. 2, (A) to (I) are as follows:
(A) Absorbance spectra of blank bEVs, Ce6, 2-hydroxyethyl TPP, TPP-Ce6, and bEV(TPP-Ce6) solutions.
(B) TEM images of blank bEV and bEV(TPP-Ce6). Scale bars each indicate 50 nm.
(C) Changes in fluorescence intensity of TPP-Ce6 and bEV(TPP-Ce6) after incubation in PBS for 14 days under normal light conditions.
(D) TPP-Ce6 release profiles from bEV(TPP-Ce6) before and after light irradiation (660 nm, 1 min) at different incubation times.
(E) Schematic diagram of in vitro BBB model using bEnd.3 cells.
(F) Apparent permeability coefficient (Papp) of TPP-Ce6 and bEV(TPP-Ce6) measured using an in vitro BBB model.
(G) Relative Ce6 uptake of bEV(TPP-Ce6) by U87MG cells after crossing BBB layers.
(H) Relative cellular uptake of bEV(TPP-Ce6) in U87MG cells following pre-treatment with Tf at different concentrations.
(I) Changes in cellular uptake level of bEV(TPP-Ce6) by different cell lines after pre-treatment with Tf (100 μg/mL).

In FIG. 3, (A) to (E) are as follows:
(A) Relative mitochondrial uptake of Ce6, TPP-Ce6, and bEV(TPP-Ce6) in U87MG cells.
(B) Confocal micrographs displaying intracellular localization of Ce6 and TPP-Ce6 in U87MG cells. U87MG cells were treated with Ce6 or TPP-Ce6 (red) for 2 h, then stained with MitoTracker (green) to visualize mitochondria. The red fluorescence intensity of the cells and Mander's overlap coefficients (indicating colocalization degrees of red and green) were calculated from the images. Scale bars each indicate 20 μm.
(C) Intracellular localization of rhodamine B (RB)-labeled bEV(TPP-Ce6) in U87MG cells stained with MitoTracker Green. Mander's overlap coefficients indicating the RB-labeled bEVs or TPP-Ce6 signals overlapped with mitochondria staining were determined from the images. Scale bars each indicate 25 μm.
(D) Relative intracellular ROS levels in U87MG cells treated with Ce6, TPP-Ce6, and bEV(TPP-Ce6) before and after light irradiation (660 nm, 1 min).
(E) Fluorescence images of U87MG cells generating intracellular ROS after being treated with Ce6, TPP-Ce6, and bEV(TPP-Ce6) in the absence or presence of light exposure (660 nm, 1 min). Scale bars each indicate 50 μm.

FIG. 4A is fluorescence images of JC-1 stained U87MG cells after various treatments: 1) control, 2) Ce6+L, 3) TPP-Ce6+L, and 4) bEV(TPP-Ce6)+L. The red JC-1 aggregates indicate mitochondria with normal membrane potential, and the green JC-1 monomer indicates mitochondria with a depolarized membrane. Red/green fluorescence ratios of JC-1 stained U87MG cells after various treatments were obtained from the fluorescence images.

FIG. 4B is a diagram showing viabilities of U87MG cells treated with Ce6, TPP-Ce6, and bEV(TPP-Ce6) before and after light irradiation (660 nm, 1 min).

FIG. 4C is fluorescence images of co-stained U87MG cells after treatment with Ce6, TPP-Ce6, and bEV(TPP-Ce6) before and after light irradiation (1 min).

FIG. 4D is a diagram showing viabilities of U87MG cells treated with various concentrations of bEV.

FIG. 4E is a result of apoptosis staining of U87MG cells treated with Ce6, TPP-Ce6, and bEV(TPP-Ce6).

FIG. 5A is in vivo real-time fluorescence imaging of an orthotopic GBM-xenografted mouse model after i.v. administration of free Ce6, free TPP-Ce6, and bEV(TPP-Ce6). Luminescence imaging of mice before injection with samples was also conducted to determine the location of brain tumors.

FIG. 5B is a diagram showing time course ratios of the fluorescence intensity in the whole brain at each time point to the whole body at 1 min for free Ce6, free TPP-Ce6, and bEV(TPP-Ce6)-treated mice (n=4), determined by IVIS fluorescence imaging.

FIG. 5C is a diagram showing time course ratios of the fluorescence intensity in the brain tumor to the whole brain for free Ce6, free TPP-Ce6, and bEV(TPP-Ce6)-treated mice (n=4), determined by IVIS fluorescence imaging.

FIG. 5D is ex vivo imaging of tumor-containing brain and major organs at 24 h post-injection of free Ce6, free TPP-Ce6, and bEV(TPP-Ce6).

FIG. 5E is a diagram showing quantitation of a fluorescence signal from ex vivo imaging of tumor-containing brain and major organs at 24 h-post i.v. administration of free Ce6, free TPP-Ce6, and bEV(TPP-Ce6).

FIG. 5F is confocal micrographs showing the distribution of FITC-labeled bEV(TPP-Ce6) in the brain and brain tumors of mice after i.v. injection. The boundary between tumor regions and surrounding normal tissue is indicated by a white dotted line. Each scale bar indicates 200 μm.

FIG. 5G is diagrams showing quantitation of mean fluorescence intensities from FITC-bEV and TPP-Ce6 accumulated in tumor regions and surrounding normal tissue in the brains of mice after i.v. injection with FITC-labeled bEV(TPP-Ce6).

FIG. 6A is imaging of a time-lapse bioluminescence intensity (BLI) in the brain tumors of orthotopic GBM-xenografted mice after i.v. administration with PBS, free Ce6, free TPP-Ce6, and bEV(TPP-Ce6) upon light irradiation (660 nm, 5 min). The mice were irradiated with 660 nm light for 5 min at 4 h post i.v. injection of each sample.

FIG. 6B is a diagram showing changes in the BLI of brain tumors of mice after treatment with various samples and light irradiation compared to that on day 0 (n=4).

FIG. 6C is a diagram showing changes in the body weights of GBM-xenografted mice (n=4) for 9 days after i.v. injection of various samples and light irradiation (n.s.; not significant).

FIG. 6D is an image of H&E stained brain sections of mice on day 9 after i.v. injection of PBS, Ce6, TPP-Ce6, and bEV(TPP-Ce6) following light irradiation. Tumor regions of interest in the brain section (indicated by black boxes) are magnified. Scale bars of the magnified images each indicate 100 μm.

FIG. 6E is H&E staining images of major organs of the mice on day 9 after i.v. injection of various samples and light irradiation. Scale bars each indicate 100 μm.

FIG. 7 is a ¹H NMR spectrum of TPP-Ce6. The multiple peaks at 7.7-7.9 ppm indicate that TPP [(C₆H₅)₃P⁺] was successfully conjugated to Ce6.

FIG. 8 is a diagram showing size changes of bEV(TPP-Ce6) incubated in PBS-containing 10% FBS for 5 days, determined by NTA.

FIG. 9 is a diagram showing apparent permeability coefficients (Papp) of bEV and bEV(TPP-Ce6) in an in vitro BBB double layer model. bEVs were labeled with rhodamine B.

FIG. 10 is a diagram showing transferrin concentration of bEVs at different EV concentrations.

FIG. 11 is confocal micrographs displaying intracellular localization of RB-labeled bEV(TPP-Ce6) in bEnd.3 and U87MG cells. Both bEnd.3 and U87MG cells were treated with RB-labeled bEV(TPP-Ce6) (red) for 2 h, then stained with LysoTracker (green). Mander's overlap coefficients (indicating colocalization degrees of red and green) were calculated from the images. Scale bars each indicate 20 μm.

FIG. 12 is a diagram showing a relative ROS level in a cell-free system treated by Ce6 and TPP-Ce6.

### DETAILED DESCRIPTION OF THE DISCLOSURE

Glioblastoma (GBM) is the most aggressive type of malignant brain tumor and patients with GBM have a high mortality rate. Photodynamic therapy (PDT) has emerged as a promising approach for the treatment of malignant brain tumors. However, the use of PDT for the treatment of GBM has been limited because its ability to permeate the blood-brain barrier (BBB) is weak and its ability to target cancer is lacking.

The present inventors used brain vascular endothelial cell-derived extracellular vesicles (bEVs) as a drug delivery system for penetrating the BBB. Ce6 (chlorine e6) was used as a photosensitizer to improve PDT efficacy, and tryphenylphosphonium (TPP) was conjugated on Ce6 to impose mitochondrial targeting ability and then loaded onto the bEV. TPP-Ce6 was selectively accumulated in mitochondria and was more effective in inducing mitochondrial damage and apoptosis by ROS generation under light irradiation. In addition, loading TPP-Ce6 into the bEV, remarkably improved the aqueous stability and cellular uptake of TPP-Ce6, and it was confirmed that the increase in cellular uptake significantly increased PDT efficacy. Specifically, TPP-Ce6 effectively accumulated in the mitochondria of U87MG human GBM cells and showed higher PDT efficacy than Ce6. Importantly, bEVs greatly enhanced the aqueous stability and cellular uptake of TPP-Ce6, which led to enhanced intracellular ROS generation under laser irradiation. Therefore, bEV(TPP-Ce6) showed significantly enhanced PDT performance in U87MG cells. An in vivo biodistribution study using orthotopic GBM-xenografted mice showed that bEV(TPP-Ce6) effectively targeted brain tumors after efficient BBB penetration. As a result, treatment with bEV(TPP-Ce6) and light irradiation efficiently inhibited tumor growth in GBM-xenografted mice without causing adverse systemic toxicity. This study offers new insights into the use of biocompatible bEVs to make photodynamic GBM therapy safer and more efficient.

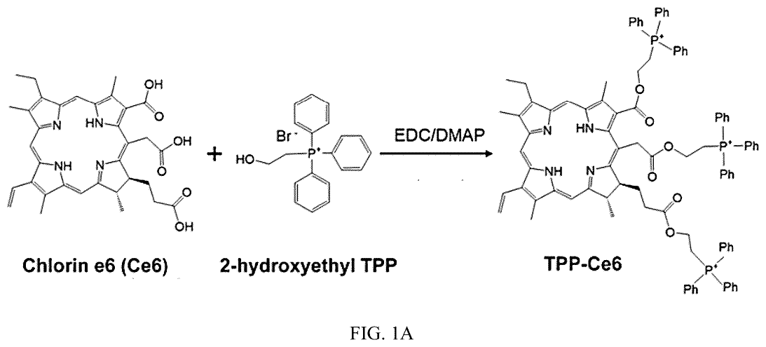

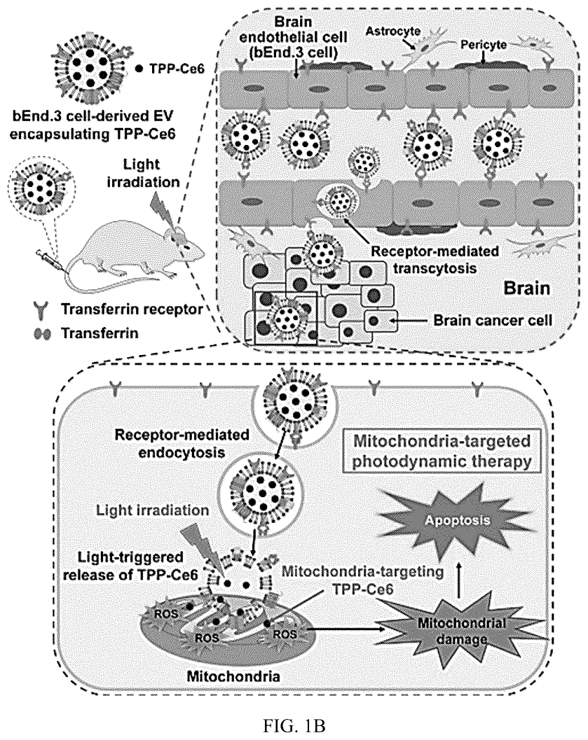

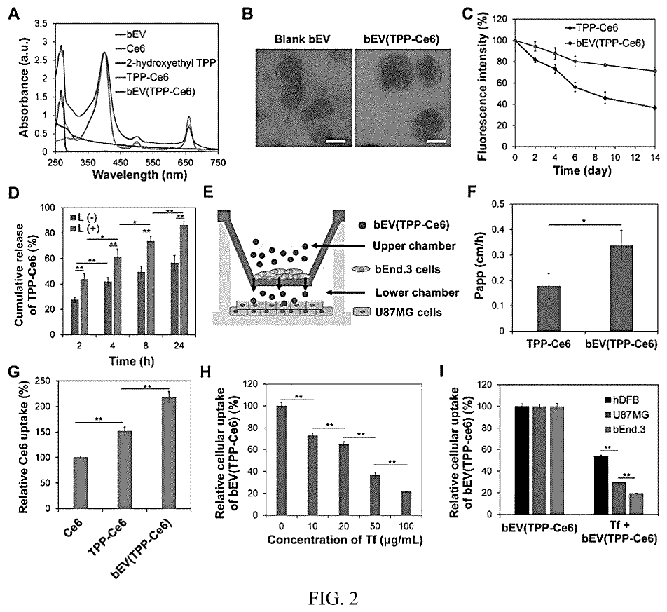

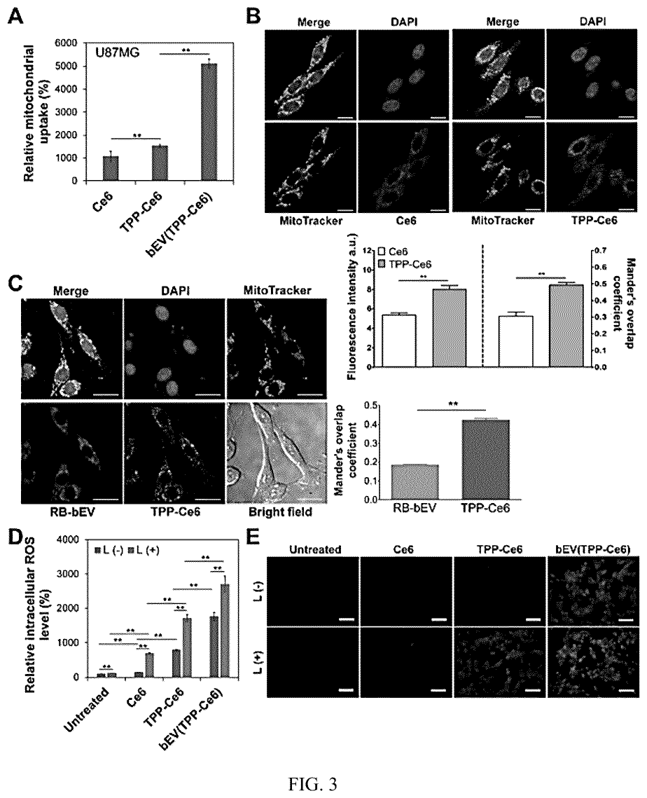

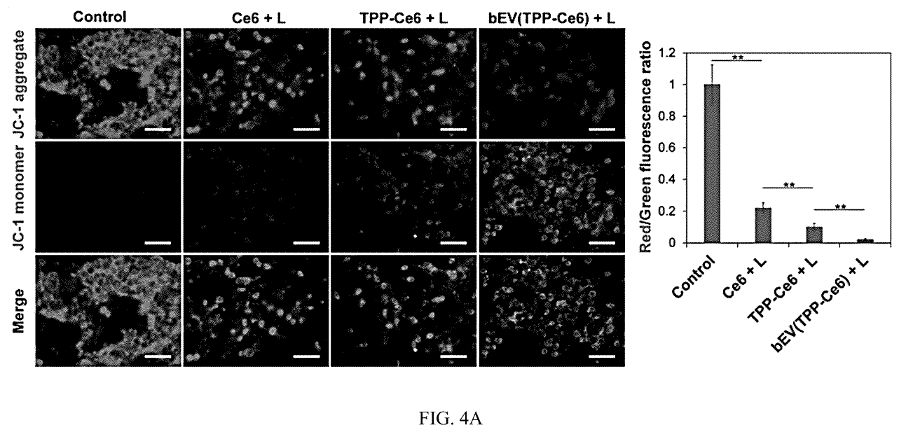

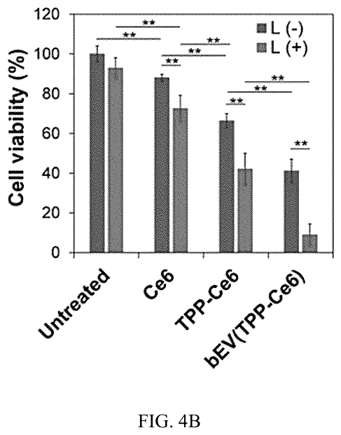

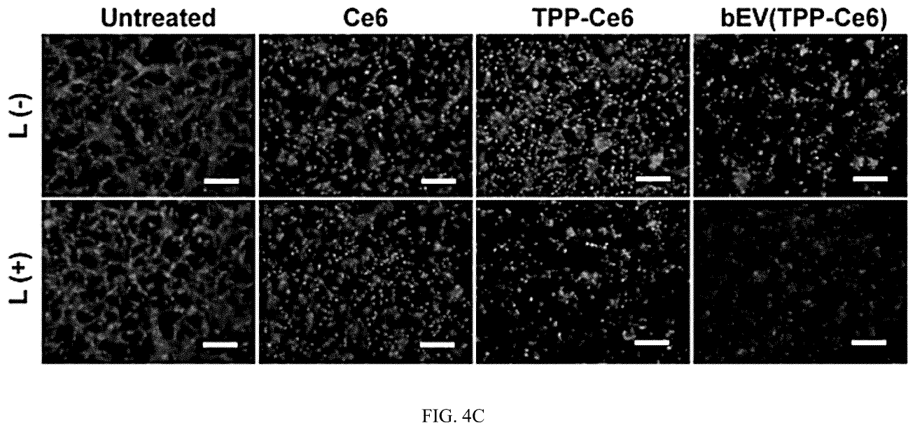

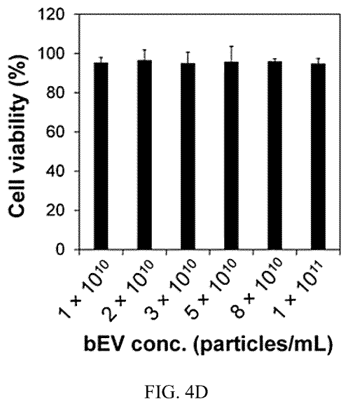

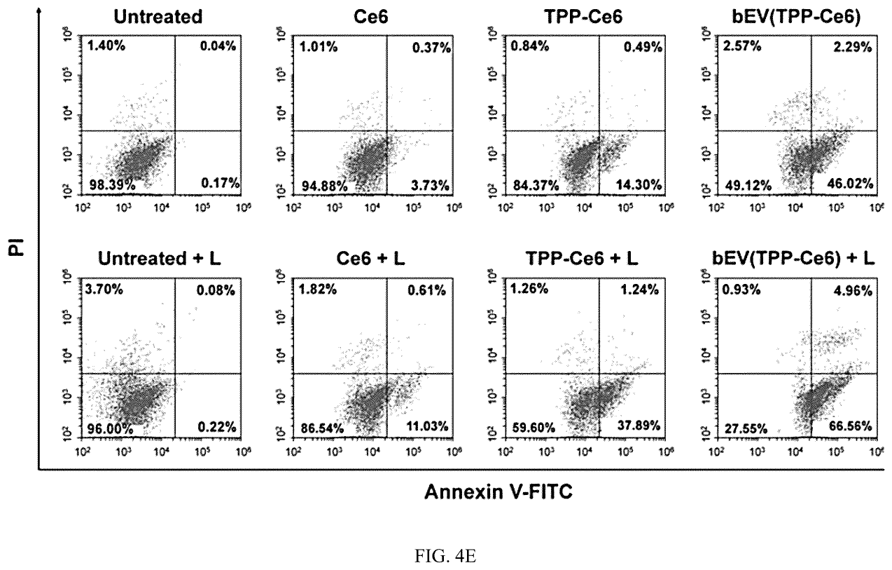

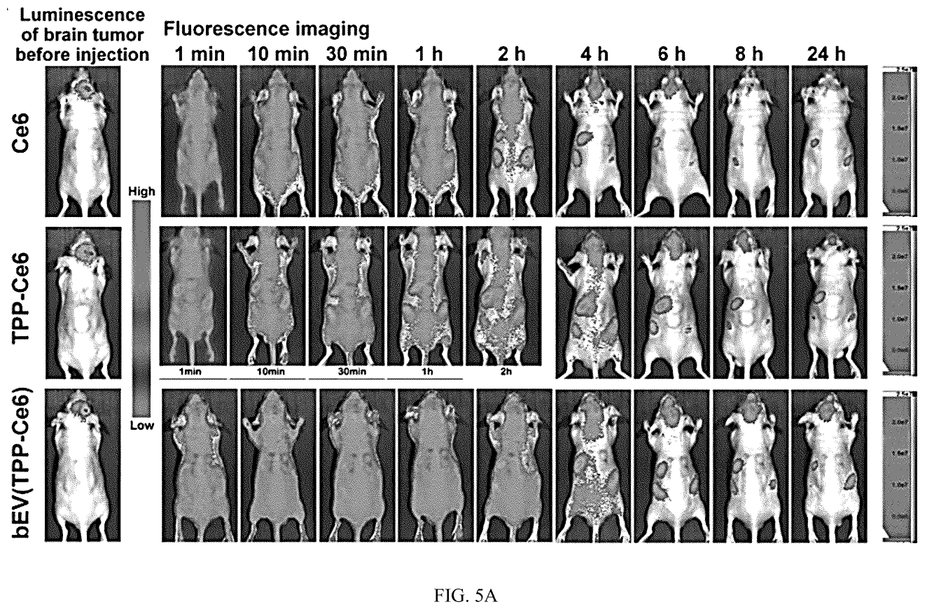

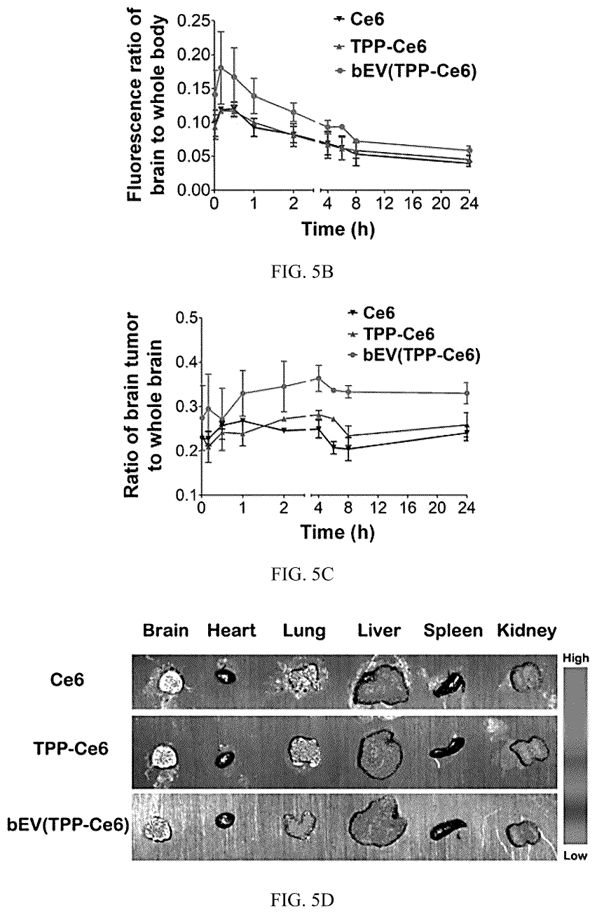

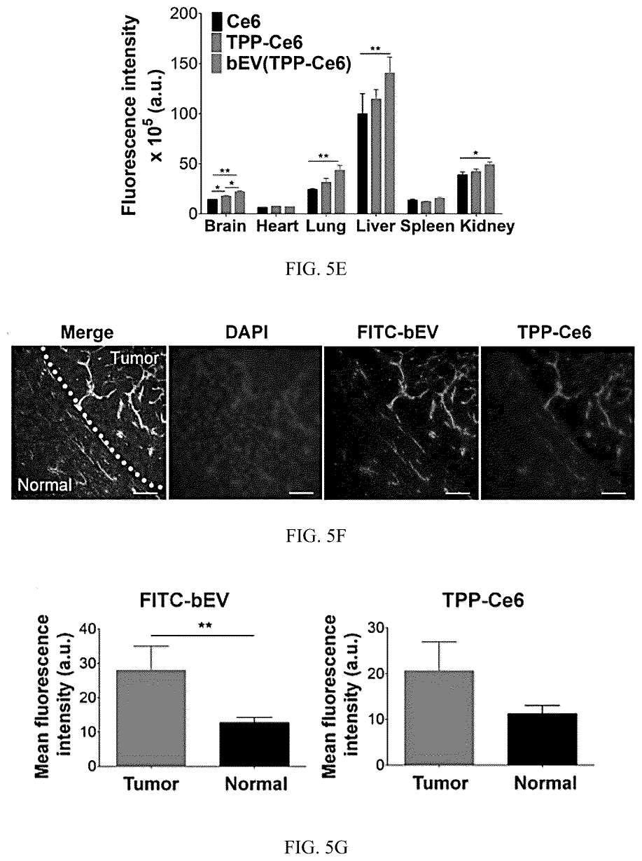

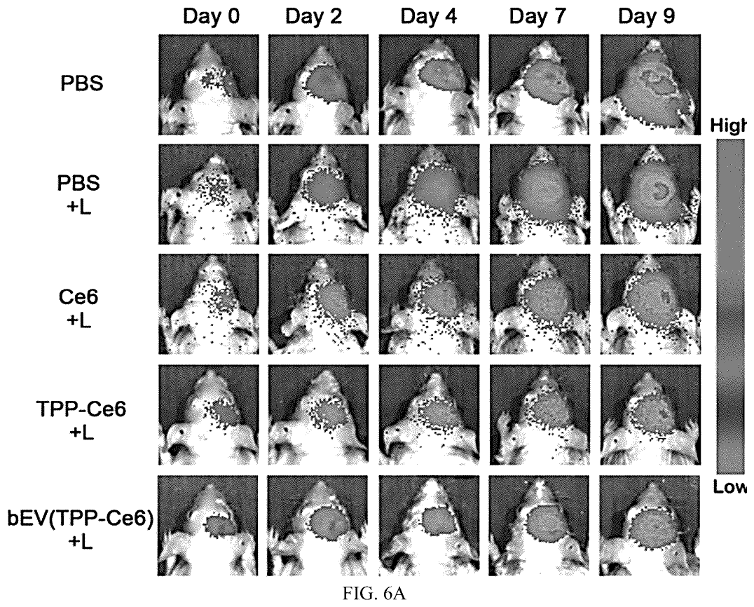

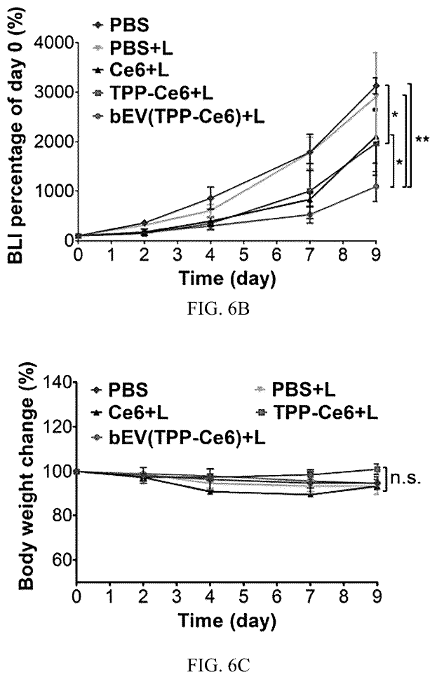

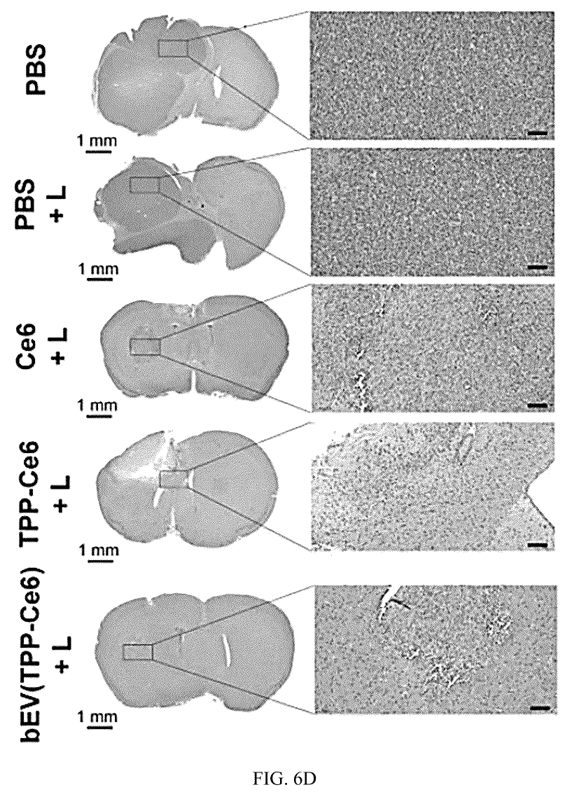

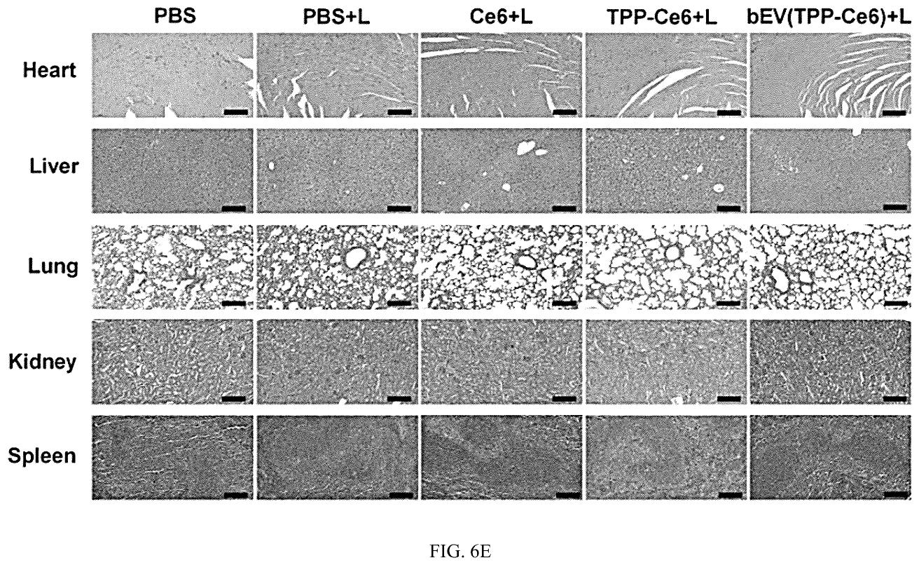

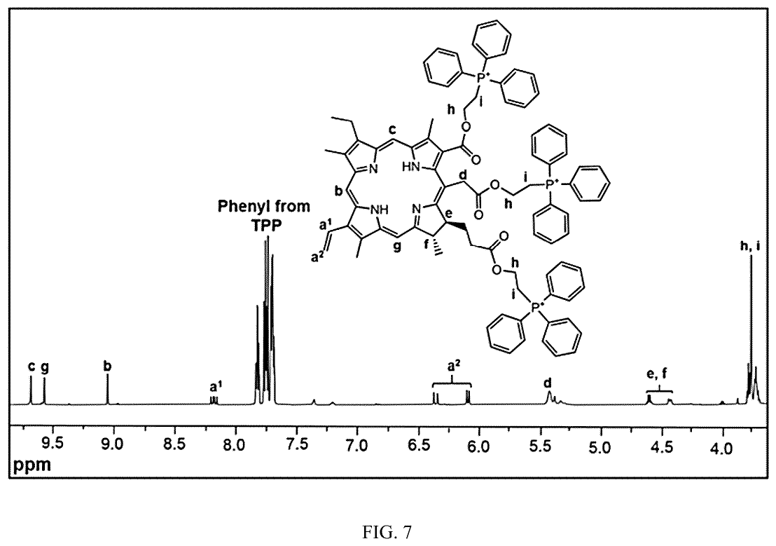

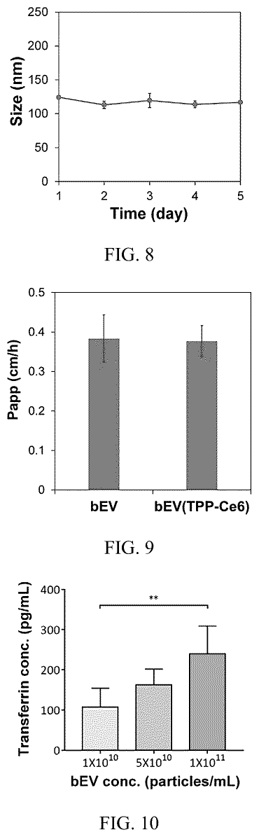

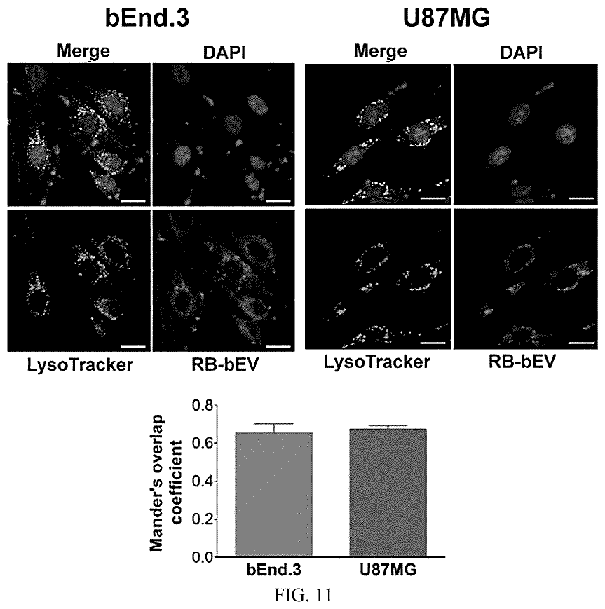

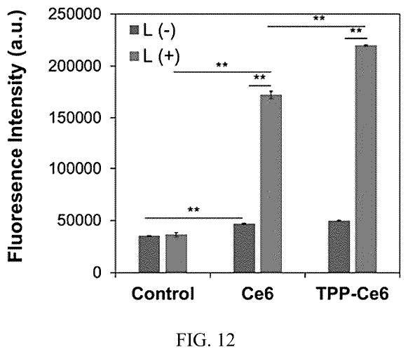

FIG. 1B is a schematic view of a PDT using the bEV equipped with TPP-Ce6 having the mitochondrial targeting ability of the present disclosure. bEV (TPP-Ce6) administered to a brain tumor transplanted mouse, penetrates the BBB through a receptor mediated transcytosis and is introduced into brain tumor cells through endocytosis. After light exposure, TPP-Ce6 is released from the bEV, and the released TPP-Ce6 is preferentially accumulated into the mitochondria and generates ROS during light irradiation. ROS induces mitochondrial damage and apoptosis. The biocompatible bEV-based mitochondrial targeting photosensitizer of the present disclosure may be applied to the treatment of glioblastoma in clinical practice.

In the present specification, the term “extracellular vesicle(EV)” means a nano-sized particle that is naturally secreted by being packaged in a lipid bilayer in a living cell. Conceptually, the EV may include all endoplasmic reticulums (exosomes, microspheres, multicelles, and extracellular endoplasmic reticulum-like endoplasmic reticulum) that serve as cell-to-cell information transmission.

In the present specification, “isolated extracellular vesicles” means cells from which extracellular vesicles originate, for example, extracellular vesicles substantially separated from brain vascular endothelial cells.

In one embodiment, the extracellular vesicles may be derived from the same species as the subject to which the drug carrier system is applied, may be brain vascular endothelial cells derived from humans or mice, specifically secreted from hCMEC/D3 or bEnd.3 cells, and may be isolated from the culture medium of the cells.

The extracellular vesicles of the present disclosure are derived from brain vascular endothelial cells and may penetrate a transferrin receptor-mediated blood-brain barrier (BBB). In addition, the extracellular vesicles are selectively taken up by brain tumor cells and may be provided as a pharmaceutical composition for brain tumor treatment including one or more known anticancer drugs inducing apoptosis, and may be used as a drug composition further including both a photosensitizer and the one or more anticancer drugs.

In the present specification, the term “photosensitizer” means a molecule that is activated by light of a specific wavelength and reacts with oxygen molecules around it to generate ROS, and in an embodiment, the photosensitizer may be Ce6.

In the present disclosure, the photosensitizer could be conjugated with a mitochondria targeting molecule, and in an embodiment of the present disclosure, the mitochondria targeting molecule could be triphenylphosphonium (TPP), but is not limited thereto. In the present disclosure, the TPP-conjugated photosensitizer is a photosensitizer covalently linked with TPP.

In the present disclosure, “Treatment” means any action whereby the symptoms of the tumor (cancer) are reduced or changed to a beneficial effect via administration of the composition of the present disclosure. In the present disclosure “Prevention” means any action that inhibits or delays the occurrence, metastasis, or recurrence of a tumor (cancer) via administration of the composition of the present disclosure. In the present disclosure “Effect enhancement” means that the intended effect of using the photosensitizer is better when the photosensitizer is loaded on the bEV than when the photosensitizer is used alone. The intended effect of using a photosensitizer in the present invention is inducement of ROS production in brain tumor cells, especially in mitochondria among organelles of the brain tumor cell.

In the present disclosure, “pharmaceutical composition” means a product produced for the purpose of preventing or treating a disease, and the product may be produced in various forms according to a conventional method. For example, it may be produced in forms for oral administration such as powders, granules, tablets, capsules, suspensions, emulsions, and syrups, and may be produced in the forms of external preparations, suppositories, and sterile injection solutions. Further, according to each form, pharmaceutically acceptable carriers, such as buffers, preservatives, analgesics, solubilizers, isotonic agents, stabilizers, bases, excipients, lubricants, etc. which are well known in the art may be added to the composition. On the other hand, the pharmaceutical composition according to the present disclosure may be administered in a pharmaceutically effective amount. In the present disclosure, the term “pharmaceutically effective amount” means an amount at a level that is sufficient to treat a disease with a reasonable benefit/risk ratio applicable to medical treatment and which will not cause side effects. The effective dose level may be determined according to factors including the patient's health status, severity, drug activity, drug sensitivity, administration method, administration time, administration route and excretion rate, duration of treatment, combination or concurrent drugs, and other factors well-known in the medical fields. Therefore, administering the pharmaceutical composition according to the present disclosure to a subject may prevent or treat cancer (tumor), and enhance the effect of anticancer treatment, especially PDT.

In the present disclosure, “subject” means a mammal, such as a rat, livestock, mouse, or human, preferably a human. The pharmaceutical composition according to the present disclosure may be produced in various forms for administration to a subject, and a representative form for parenteral administration is an injectable form, preferably an isotonic aqueous solution or suspension. Injectable forms may be produced by techniques known in the art using suitable dispersing or wetting agents and suspending agents. For example, each component may be dissolved in saline or buffer such that it is in injectable form. Further, the pharmaceutical composition according to the present disclosure may further include adjuvants such as preservatives, wetting agents, emulsification accelerators, salts or buffers for regulating osmotic pressure, and other therapeutically useful materials, and may be formulated by conventional methods. The pharmaceutical composition according to the present disclosure may be administered intravenously. The dosage of the active ingredient may be appropriately selected based on several factors such as the route of administration, age, sex, weight of patient and a severity thereof. Further, the composition according to the present disclosure may be administered in parallel with a known compound that may enhance the intended effect.

The present disclosure may be modified and exemplified in various ways. Thus, hereinafter, specific examples are illustrated in the drawings and described in detail in the detailed description. However, the intention is not to limit the present disclosure to specific examples. The disclosure should be understood as including all variations, equivalents to, and substitutes included in the spirit and scope of the present disclosure. When it is determined that a detailed description of a related known step or element may obscure the gist of the present disclosure, such detailed description will be omitted from the concerned description of the present disclosure.

### Material and Method

### 1. Material

(2-hydroxyethyl)triphenylphosphonium bromide (2-hydroxyethyl TPP), 2′,7′-dichlorofluorescein diacetate (DCF-DA), 1-ethyl-3-(3′-dimethylaminopropyl)carbodiimide

(EDC)·HCl, 4-dimethylaminopyridine (DMAP), and 3-(4,5-dimethyl-2-thiazolyl)-2,5-diphenyltetrazolium bromide (MTT) were supplied by Sigma-Aldrich (St. Louis, MO, USA). Ce6 was supplied by Hangzhou Dayangchem Co. Ltd. (Hangzhou, China). Slide-A-Lyzer Mini Dialysis devices (MW cutoff=20 kDa) were purchased from Thermo Fisher Scientific (Waltham, MA, USA). Dulbecco's modified Eagle's medium (DMEM) and fetal bovine serum (FBS) were supplied by HyClone (Cytiva).

### 2. Synthesis of TPP-Conjugated Ce6

To prepare TPP-Ce6, 100 mg of Ce6 (0.168 mmol, 1 equiv.) was mixed with 128.8 mg of EDC·HCl (0.672 mmol, 4 equiv.) and 82.1 mg of DMAP (0.672 mmol, 4 equiv.) in 5 mL dimethylforamide (DMF). The reaction mixture was allowed to stir at room temperature for 2 h in the dark. Then, 324.3 mg of 2-hydroxyethyl TPP (0.84 mmol, 5 equiv.) in 2 mL of DMF was added to the above-mentioned mixture and the mixture was stirred for 24 h at room temperature. The crude product was precipitated using cold diethyl ether and purified by silica gel column chromatography. Eluents with a gradient from dichloromethane to 80/19/1 dichloromethane/methanol/triethylamine were used to obtain the product in the form of a dark green solid (150 mg, 0.102 mmol, 60.7% yield). The structure of TPP-Ce6 was confirmed by ¹H NMR (FIG. 7).

3. Preparation and Characterization of bEVs and TPP-Ce6-Loaded bEVs

bEnd.3 cells were cultured in DMEM with EV-free FBS (Thermo Fisher Scientific) to produce EVs. bEnd.3 cell-cultured media were centrifuged for 15 min at 1,500 g to remove cell debris. bEVs were isolated using ExoQuick-TC™ (System Biosciences, Palo Alto, USA). Nanoparticle tracking analysis (NTA, Nanosight NS300, Malvern, Worcestershire, UK) was used to determine the concentration of bEVs. To produce TPP-Ce6-loaded EVs, high concentration stock solutions of TPP-Ce6 (20 mg/mL) were prepared in dimethyl sulfoxide (DMSO). 5 μL of TPP-Ce6 solution in DMSO (0.2 mg/mL) was incubated with 5×10¹¹ EVs in 0.5 mL PBS. The DMSO concentration in the PBS was adjusted to 4% (v/v). The solution was subjected to agitation for 2 h. Subsequently, the mixture was purified of free TPP-Ce6 by PD G-25 desalting columns (Cytiva), resulting in bEV(TPP-Ce6). The bEV(TPP-Ce6) was stored at 4° C. for further use. Successful loading of TPP-Ce6 into the bEVs was confirmed by US-Vis spectroscopy (AquaMate 8100, Thermo Fisher Scientific). The absorbance of TPP-Ce6 at 664 nm was measured to calculate the encapsulation efficiency of TPP-Ce6 in EVs, which is defined as follows: (weight of loaded TPP-Ce6)/(weight of initially added TPP-Ce6)×100%. The hydrodynamic sizes and surface charges of various EV-derived samples were measured using NTA (Nanosight NS300) and Zetasizer (Nano-ZS, Malvern), respectively. Morphologies of blank bEVs and bEV(TPP-Ce6) were observed by transmission electron microscopy (TEM, Talos F200X, FEI, Hillsboro, OR, USA).

4. Assessment of Colloidal Stability and Long-Term Aqueous Stability of bEV(TPP-Ce6)

The colloidal stability of bEV(TPP-Ce6) against serum proteins was assessed by incubating them in 10% FBS-containing PBS at 37° C. for 5 days in the dark. The samples were collected every day to monitor the size changes of bEV(TPP-Ce6) during the incubation period. The size of bEV(TPP-Ce6) was measured using NTA (Nanosight NS300). To examine long-term aqueous stability of TPP-Ce6 and bEV(TPP-Ce6), the samples were prepared in PBS (10 μg/mL) and incubated at 4° C. in normal light conditions. At predetermined time points, the fluorescence intensities of TPP-Ce6 and bEV(TPP-Ce6) (λex=410 nm: λem=660 nm) were quantified using a spectrophotometer (SpectraMax® iD3, Molecular Devices, San Jose, CA, USA).

5. In Vitro Drug Release Profiles of bEV(TPP-Ce6) Under Light Irradiation

Cumulative amounts of TPP-Ce6 released from bEV(TPP-Ce6) with or without laser (660 nm) treatment were quantified using dialysis membranes. Approximately 100 μL of bEV(TPP-Ce6) suspension (10 μg/mL TPP-Ce6) was loaded into a Slide-A-Lyzer Mini Dialysis device. To investigate drug release from bEV(TPP-Ce6) in response to light, the dialysis devices containing the samples were irradiated with a 660-nm laser (150 mW, beam diameter of 5 mm) for 1 min. The sample not subjected to laser treatment was adopted as a control. After light exposure, the dialysis devices were incubated at 37° C. while being agitated in the dark. At certain predetermined time intervals (i.e., 2, 4, 8, and 24 h), the sample solutions were collected following a previously reported protocol31. Amounts of TPP-Ce6 released from the samples were quantified by the measurement of its absorbance at 664 nm.

### 6. Enzyme-Linked Immunosorbent Assay (ELISA)

Standard and EV samples were added to ELISA well and incubated for 1 h at 37° C. The transferrin concentrations were quantified using a Mouse Transferrin Sandwich ELISA kit (LSBio, USA), according to the manufacturer's protocol. The optical density (OD value) of the samples was measured at 450 nm.

### 7. In Vitro Transcytosis of TPP-Ce6-Loaded EVs Using a Transwell BBB Model

The in vitro BBB model composed of bEnd.3 and U87MG cells was constructed to assess the BBB permeability and glioma uptake of bEV(TPP-Ce6). bEnd.3 and U87MG cells were cultured in 10% FBS-containing DMEM. The simulated BBB double-layer was prepared by culturing bEnd.3 cells on transwell filter inserts (0.4-μm pore size, Corning-Costar Corp., Corning, USA) and U87MG cells in the lower compartment. Briefly, bEnd.3 cells in 400 μL of culture media were seeded on the transwell filter inserts (5×10⁶ cells/well). U87MG cells in 800 μL of culture media were inoculated in the lower chamber (5×10⁶ cells/well). The culture media were replaced every two days, and the integrity and permeability of the cell layers were assessed by way of measuring trans-epithelial electrical resistance (TEER) using an EVOM2 epithelial voltohmeter (World Precision Instruments Inc., Sarasota, USA). The multilayered bEnd.3 cells were cultured until their TEER values reached 200 Ω·cm² at least. After the culture medium in the upper chamber was removed, TPP-Ce6 or bEV(TPP-Ce6) (10 μM TPP-Ce6) in DMEM was added into the compartment, then incubation was performed for 4 h. To quantify the transcytosis of bEV(TPP-Ce6) across the BBB layers, the amount of TPP-Ce6 in the lower chamber was quantified by way of measuring its fluorescence using a spectrophotometer (SpectraMax® iD3). To quantify the cell uptake of Ce6, TPP-Ce6, and bEV(TPP-Ce6) by U87MG cells in the stimulated BBB model, U87MG cells cultured in the lower compartment were collected at 4 h post-incubation with the samples (10 μM of Ce6 and TPP-Ce6). The amount of Ce6 or TPP-Ce6 in the cells was analyzed using a flow cytometer (CytoFLEX, Beckman Coulter, Brea, CA, USA). The apparent permeability coefficients (Paap) of TPP-Ce6 and bEV(TPP-Ce6) were calculated using the following formula:

$${P}_{app} = \frac{{C}_{receiver} \times {V}_{receiver}}{A \times t \times {C}_{donor initial}} \qquad [ formula 1 ]$$

A: membrane area (0.33 cm²);
t: incubation time (4 h);
V_{receiver}: volume of receiver (lower compartment, cm³);
C_{receiver}: concentration of receiver (lower compartment, μg/mL);
C_{donor intitial}: initial concentration oBf donor (upper compartment, μg/mL).

To quantify the bEVs penetrating the simulated BBB layers, fluorescently labeled bEVs were used to encapsulate TPP-Ce6. Rhodamine B (RB)-labeled bEVs (RB-bEVs) were prepared by mixing 1×10¹² bEVs with 0.4 μg RB isothiocyanate (Sigma-Aldrich) in 0.5 mL PBS. The solution was stirred at 4° C. for 2 h in the dark. It was subjected to PD G-25 columns to remove free RB isothiocyanate. RB-bEVs or RB-labeled bEV(TPP-Ce6) (10 μM TPP-Ce6) in DMEM were added into the upper compartment as described above. After 4 h of incubation, the amounts of RB-bEV and RB-labeled bEV(TPP-Ce6) that crossed the simulated BBB were determined by measuring the fluorescence intensity of RB in the cells cultured in each compartment. Papp values of the samples were also calculated based on the aforementioned formula 1.

8. Mechanism of Transferrin Receptor-Mediated Transcytosis of bEV(TPP-Ce6)

To examine the mechanism behind the transcytosis of bEV(TPP-Ce6) across the BBB, hDFB, U87MG, and bEnd.3 cells were inoculated in a 12-well plate at a density of 3×10⁵ cells/well for 24 h. Then, the cells were washed by PBS. Subsequently, the cells were pretreated with transferrin (apo-transferrin from mouse, Sigma-Aldrich) at various concentrations. After 30 min of treatment, the cells were rinsed with PBS and incubated with bEV(TPP-Ce6), then incubated for 4 h. The cells not subjected to pre-treatment with transferrin were also incubated with bEV(TPP-Ce6) as a control. The cells were detached by trypsin, and their fluorescence intensity was quantified by flow cytometry (CytoFLEX).

9. Mitochondrial Uptake Analysis of Ce6, TPP-Ce6, and bEV(TPP-Ce6)

U87MG cells were cultured in 6 well-plates at a density of 1×10⁶ cells/well. After 1 day of incubation, Ce6, TPP-Ce6, and bEV(TPP-Ce6) at an equivalent TPP-Ce6 (or Ce6) concentration of 10 μM were added to the cells, then incubation was performed for 4 h. Mitochondrial and cytosolic fractions from the cells were collected using a Mitochondria/Cytosol Fractionation Kit (BioVision, Milpitas, CA, USA). The relative mitochondrial uptake levels of Ce6 (or TPP-Ce6) for each sample [i.e., [mitochondrial uptake of Ce6 (or TPP-Ce6)]/[total cellular uptake of Ce6 (or TPP-Ce6)]×100%] were determined using a flow cytometer (CytoFLEX).

### 10. In Vitro Intracellular Localization Study by Confocal Laser Scanning Microscopy (CLSM)

U87MG and bEnd.3 cells (2×10³ cells/well) were prepared in glass-bottom dishes (35×10 mm²) and incubated for 24 h. Free Ce6, free TPP-Ce6, and bEV(TPP-Ce6) in serum-free medium were added to the cells at a final TPP-Ce6 (or Ce6) concentration of 0.15 μM. After 2 h of incubation at 37° C., the cells were washed three times with PBS and stained with 1 μg/mL DAPI (Thermo Fisher Scientific) for 10 min. Then, the cells were stained with 1 μM of LysoTracker Green DND-26 (Thermo Fisher Scientific) and 500 nM of MitoTracker Green FM (Thermo Fisher Scientific) for endo/lysosome and mitochondria staining, respectively. The glass-bottom dish was placed in live-cell chamber systems (5% CO₂ at 37° C.) connected to a CLSM (Al Plus, Nikon, Japan). The obtained fluorescent images were analyzed for the quantification of colocalization ratios.

11. ROS Generation by bEV(TPP-Ce6) Upon Light Irradiation

The generation of ROS by Ce6 and TPP-Ce6 before and after 660 nm laser treatment was quantified using 2′,7′-dichlorofluorescein (DCF) in a cell-free system. After the sample solutions were treated with DCF for 1 h in the dark, ROS generation was determined by measuring the fluorescence signal from the samples. The intracellular ROS production in U87MG cells after various treatments was measured using DCF-DA. U87MG cells were inoculated into 12 well-plates at a density of 3×10⁵ cells/well and cultured overnight. After the cells were incubated with 10 μM DCF-DA for 15 min, they were treated with Ce6, TPP-Ce6, and bEV(TPP-Ce6) at a TPP-Ce6 (or Ce6) concentration of 10 μM. After 4 h of incubation, the cells were trypsinized and divided into two groups. One of the groups was treated with a 660 nm laser for 1 min. The cells not subjected to laser treatment were kept as a control. After 30 min of incubation in the dark, relative ROS generation levels of each sample were quantified by flow cytometry (CytoFLEX).

To visualize ROS generation by TPP-Ce6 and bEV(TPP-Ce6) in U87MG cells under light irradiation, the cells were inoculated into 12-well plates (3×10⁵ cells/well) and stained with 10 μM of DCF-DA. Ce6, TPP-Ce6, and bEV(TPP-Ce6) (10 UM of Ce6 or TPP-Ce6) were added to the DCF-DA-stained cells, then incubation was performed for 4 h. Then, the cells were rinsed with PBS. For the light-treated samples, the cells were irradiated with a 660 nm laser for 1 min, then incubated for 30 min. The green fluorescence signals indicating intracellular ROS were observed using a fluorescence microscope (Nikon Eclipse Ti-S, Nikon, Tokyo, Japan).

### 12. JC-1 Staining Assay

Changes in the mitochondria membrane potentials of U87MG cells after various treatments were evaluated using a conventional JC-1 staining assay. U87MG cells were cultured in 12-well plates (3×10⁵ cells/well) 24 h before treatment with Ce6, TPP-Ce6, and bEV(TPP-Ce6) (10 UM of Ce6 or TPP-Ce6). After 4 h of incubation, the cells were exposed to a 660-nm laser for 1 min. The untreated cells were adopted as a control. Then, the irradiated cells were treated with JC-1 solution in PBS (2.5 μg/mL) for 20 min in the dark, then rinsed twice with PBS. Fluorescent images of the cells were obtained by fluorescence microscopy (Nikon Eclipse Ti—S). Red fluorescence signals indicate the JC-1 aggregates in polarized mitochondria of healthy cells whereas green fluorescence signals reflect the JC-1 monomers in depolarized mitochondria, which indicates the loss in mitochondrial membrane potential, an indicator of mitochondrial damage.

### 13. In Vitro Phototoxicity Evaluation

To investigate the photodynamic effects of TPP-Ce6-loaded EVs, the viability of U87MG cells after various treatments in the presence or absence of laser irradiation was measured using a standard MTT assay. U87MG cells were treated with Ce6, TPP-Ce6, and bEV(TPP-Ce6) at an equivalent Ce6 (or TPP-Ce6) concentration (10 μM, 5.38×10¹⁰ bEVs/mL). Cytotoxicity of blank bEVs against U87 cells was also assessed at various concentrations of bEVs. After 4 h of treatments, the cells were illuminated with a 660 nm laser for 1 min. The cells not subjected to light irradiation were adopted as a negative control. The cells were further incubated for 24 h, and their viabilities were measured.

### 14. Live and Dead Cell Staining Assay

To prepare for live and dead cell staining, U87MG cells (3×10⁵ cells/well) were separately seeded into different 12-well plates for untreated and light-treated groups. After incubation overnight, U87MG cells received Ce6, TPP-Ce6, and bEV(TPP-Ce6) (10 μM Ce6 or TPP-Ce6, 5.38×10¹⁰ bEVs/mL). After culturing for another 4 h, the cells were detached and suspended in 0.5 mL of PBS, then exposed to a 660 nm laser for 1 min. The cells were seeded back into 12-well plates and incubated for 8 h. Further, the cells were washed with PBS and stained with calcein-AM (Thermo Fisher Scientific) and ethidium homodimer-1 (Thermo Fisher Scientific) according to the manufacturer's instructions.

### 15. In Vitro Apoptosis Analysis Using Annexin V-FITC/Propidium Iodide (PI) Staining

U87MG cells (2×10⁵ cells/well) were seeded into 12-well plates and incubated overnight. The cells were treated with the TPP-Ce6 and bEV(TPP-Ce6) at an equivalent TPP-Ce6 concentration (10 μM, 5.38×10¹⁰ bEVs/mL). After 4 h of incubation, the cells were trypsinized and suspended in 0.5 mL of PBS. The cell suspensions in the microcentrifuge tubes were irradiated with 660-nm light (150 mW) for 1 min. The cells were immediately seeded back into 12-well plates and further incubated overnight. Then, the cells were detached by trypsinization and stained with Annexin V-FITC antibody and PI according to the same protocol.

### 16. Preparation of Orthotopic GBM-Xenografted Mice

All animal experiments were approved by the Institutional Animal Care and Use Committee at Gachon University (Approval number: LCDI-2017-0131). Orthotopic GBM-xenografted mice were prepared using U87MG cells stably expressing luciferase. Briefly, male athymic mice of 5 weeks of age (20-30 g) were placed on a stereotactic device (Harvard Apparatus, USA) under inhalation anesthesia using 1-1.5% isoflurane. U87MG-luc cells (1×10⁵ cells) suspended in 2 μL of PBS were injected into the right striatum at a flow rate of 0.5 μL/min through a small hole in the skull at a position 2.0 mm lateral, 0.2 mm anterior, and 3.2 mm ventral to the bregma. The formation of brain tumors in the mice was identified by bioluminescence intensity (BLI).

17. In Vivo Brain Tumor Uptake Study of bEV(TPP-Ce6)

To observe the brain tumor uptake of bEVs by CLSM, FITC-labeled bEVs were used to encapsulate TPP-Ce6. 0.4 μg of FITC was added to 500 μL PBS-containing 1×10¹² bEVs and kept at 4° C. for 2 h in the dark. The final product was purified using PD G-25 columns.

The FITC-labeled bEV(TPP-Ce6) of 100 μL was intravenously injected into the orthotopic GBM-xenografted mice through the tail vein (0.8 μmol/kg for TPP-Ce6). After 2 h, the GBM-bearing mice were transcardially perfused with 10 mM potassium PBS followed by 4% (v/v) paraformaldehyde (PFA) solution. Brains were collected, divided into 4 mm coronal slices, and post-fixed with 4% (v/v) PFA solution at 4° C. overnight. The brain coronal sections of 40 μm thickness were prepared using a vibratome (Leica V1000S, Germany) and placed on coverslips. The sections were stained by DAPI (Life Technologies) and then observed by CLSM. The fluorescence intensity in the images was quantitatively analyzed using Nikon NIS-E image analysis software.

18. In Vivo Biodistribution Study and Brain-Tumor Accumulation of bEV(TPP-Ce6) Using IVIS

To evaluate in vivo real-time biodistribution of Ce6, TPP-Ce6, and bEV(TPP-Ce6), orthotopic GBM-xenografted mice (n=4) were intravenously administered with 100 μL of each sample (0.8 μmol/kg for Ce6 or TPP-Ce6). Whole-body fluorescence images were acquired at the predetermined time points within 24 h after i.v. injection using an IVIS optical imaging system with a long wavelength emission filter (640-720 nm) (Ami HT imaging system, Spectral Instruments Imaging, Tucson, AZ, USA). Quantitative analysis of fluorescence intensity in the images was carried out using the region of interest (ROI) function of Aura Imaging 2.20 software (Spectral Instruments Imaging). The ROI of the brain tumor was manually selected based on its bioluminescence.

19. In Vivo Anti-Brain Tumor Efficacy of bEV(TPP-Ce6) with Laser Irradiation

The orthotopic GBM-bearing mice were randomly divided into five groups (n=4) and injected with one of the following regimens by i.v. injection dose of 0.8 μmol/kg Ce6 (or TPP-Ce6) via the tail vein:
(i) PBS,
(ii) PBS+L (L indicates light irradiation),
(iii) Ce6 (0.8 μmol/kg)+L,
(iv) TPP-Ce6 (0.8 μmol/kg)+L, and
(v) bEV(TPP-Ce6) (0.8 μmol/kg)+L.

At 4 h post-injection, the tumor sites of the mice were irradiated with a 660-nm laser (150 mW, 5 min) on the skull. After the injection, the bioluminescence signal from the tumor site in the mouse brain was observed daily, and a subsequent intraperitoneal injection of 150 mg/kg D-luciferin was given at each imaging session. The images were repeatedly acquired over 10 min at intervals of 2 min using an IVIS system (Ami HT imaging system). Regions of bioluminescence signal were determined using Aura imaging software (Spectral Instruments Imaging). On the ninth day after treatment with samples and light irradiation, the tumor-included brain and major organs including heart, liver, lungs, kidneys, and spleen were isolated and fixed in 4% (v/v) PFA solution for H&E staining.

### 20. Statistical Analysis

All the data was obtained in triplicate unless specified otherwise. Data is presented as mean±standard deviation (SD) values. ANOVA with Bonferroni's post-test was applied to determine the statistical significance between sample groups. The statistical significance is represented by the following: * p<0.05: ** p<0.01.

### Results

1. Design of Mitochondria-Targeting Photosensitizer-Loaded bEVs for Safe and Efficient In Vivo GBM Therapy

PDT using photosensitizer-loaded nanocarriers has great promise for the treatment of brain tumors. However, the use of photosensitizer-loaded nanocarriers for PDT of brain tumors has been limited because their ability to permeate the BBB is weak, their ability to target cancer is lacking, and nanocarriers are intrinsically toxic. Moreover, most of the artificial nanocarriers face certain critical challenges, such as systemic toxicity and poor pharmacokinetics. To overcome these challenges, we propose a naturally occurring bEV that hijacks the BBB as a biocompatible nanocarrier to transport photosensitizers into brain tumors efficiently. In addition, we propose the encapsulation of mitochondria-targeting photosensitizers in bEVs to enhance PDT efficacy for GBM through mitochondrial damage. Therefore, the mitochondria-targeting photosensitizer-loaded bEVs can achieve efficient in vivo GBM therapy owing to their intrinsic BBB-penetration capability and long-term blood circulatory capability.

2. Synthesis of TPP-Ce6 and Preparation of TPP-Ce6-Loaded bEVs

To develop a mitochondria-targeting photosensitizer, 2-hydroxyethyl TPP was chemically coupled with carboxylic acid groups of Ce6 (FIG. 1A). The synthesis of TPP-conjugated Ce6 was confirmed by ¹H NMR. As displayed in Figure S1, the peaks at 7.7-7.9 ppm indicate the presence of aromatic protons, which confirm the successful conjugation of 2-hydroxyethyl TPP to Ce6. The characteristic peaks of Ce6 were observed at 9.7, 9.6, and 9.1 ppm (═CH— in porphyrin ring) and 8.2 ppm (—CH═CH₂). The integration ratio of the peak for (C6H5)3P⁺ in the TPP moiety (7.7-7.9 ppm) to the peak for —CH═CH₂ in the Ce6 (8.2 ppm) verify that three TPP molecules were conjugated to Ce6 (FIG. 7).

To prepare TPP-Ce6-loaded EVs with enhanced BBB penetration capability, EVs were isolated from brain endothelial bEnd.3 cells, and 5×10¹¹ EVs were incubated with 0.2 mg/mL of TPP-Ce6 solution [4% (v/v) DMSO in PBS] for 2 h. Subsequently, the mixture of EVs and TPP-Ce6 was subjected to PD G-25 desalting columns to remove unloaded TPP-Ce6. Since EVs have a hydrophobic lipid bilayer surrounding a hydrophilic core18, hydrophilic TPP-Ce6 might be preferentially localized in the aqueous core or at the bilayer interface of EVs. DMSO was used to increase the solubility and loading efficiency of TPP-Ce6 owing to its permeability across lipid membranes. However, the concentration of DMSO in PBS was adjusted to 4% (v/v) because higher concentrations of DMSO induce a significant loss of EVs via membrane disruption34. The amount of TPP-Ce6 loaded into EVs was quantified by ultraviolet-visible (UV-Vis) spectroscopic analysis. FIG. 2A illustrates the UV-Vis absorption spectra of bEV, Ce6, 2-hydroxyethyl TPP, TPP-Ce6, and bEV(TPP-Ce6). The absorption spectrum of bEV(TPP-Ce6) shows distinct characteristic peaks representing 2-hydroxyethyl TPP (268 nm) and Ce6 (398 and 664 nm) (FIG. 2A). These characteristic peaks from the bEV(TPP-Ce6) absorption spectrum verify the successful encapsulation of TPP-Ce6 into bEVs. Based on the absorbance values of the TPP-Ce6 in bEV(TPP-Ce6), the loading efficiency of TPP-Ce6 in bEV(TPP-Ce6) was calculated to be 27.7%.

The hydrodynamic sizes and surface charges of blank bEVs and bEV(TPP-Ce6) were measured via nanoparticle tracking analysis (NTA) and Zetasizer, respectively. As shown in Table 1, the size of the blank bEVs was 118.0±7.3 nm and slightly increased to 122.6±7.2 nm after the loading of TPP-Ce6, which can be explained by the incorporation of lipophilic TPP-Ce6 into hydrophobic lipid bilayers of EVs. The zeta potential analysis demonstrated that all the bEV samples exhibited negative surface charges. TEM results revealed spherical morphologies of blank bEVs and bEV(TPP-Ce6) (FIG. 2B). The diameters of the blank bEVs and bEV(TPP-Ce6) were 50 to 80 nm. Using the nanoscale bEV(TPP-Ce6) prevents nonspecific clearance by the reticuloendothelial system (RES) and increases blood circulation half-life.

TABLE 1

| Samples | Size | Zeta Potential |
|---|---|---|
| bEVs | 118.0 ± 7.3 | −20.4 ± 0.8 |
| bEV(TPP-Ce6) | 122.6 ± 7.2 | −22.0 ± 1.0 |

3. High Colloidal Stability of bEV(TPP-Ce6) Under Physiological Conditions

The stability of nanoparticles in biologically relevant media plays an important role in the determining of their bioavailability in vivo36. To demonstrate colloidal stability against serum proteins, bEV(TPP-Ce6) was incubated in PBS containing 10% (v/v) EV-free fetal bovine serum (FBS) at 37° C., and size changes were monitored over 5 days. As illustrated in FIG. 8, no significant size changes were observed in the bEV(TPP-Ce6) samples. The high colloidal stability of bEV(TPP-Ce6) against serum proteins, and the nanoscale size of the vesicles should extend the half-life of the substance in circulating blood and improve tumor accumulation.

4. Long-Term Aqueous Stability of Ce6 by Encapsulation into EVs

Ce6, a fluorophore with an excellent molar extinction coefficient at 660 nm, has shown great potential as an effective therapeutic and imaging agent. However, Ce6 faces several challenges due to its inherent shortcomings, such as poor water solubility, irreversible degradation, and rapid photobleaching. To investigate whether EVs could improve the long-term aqueous stability of Ce6, the fluorescent intensities of Ce6 in TPP-Ce6 and bEV(TPP-Ce6) incubated in phosphate-buffered saline (PBS) at 4° C. were monitored for up to 14 days. As displayed in FIG. 2C, the fluorescent intensity of TPP-Ce6 markedly dropped to 36.8% of its initial value after 14 days of incubation in PBS under normal light conditions. In contrast, bEV(TPP-Ce6) retained more than 71.2% of its initial fluorescence intensity of TPP-Ce6 after incubation in PBS for 14 days. These results suggest that EVs greatly increase the stability of TPP-Ce6 in aqueous solutions by protecting it from external destabilizing radicals in solutions.

5. Light-Triggered Release of TPP-Ce6 from bEV(TPP-Ce6)

To demonstrate the light-responsive drug release of bEV(TPP-Ce6), the bEV(TPP-Ce6) was pretreated with a 660 nm laser for 1 min, then incubated in PBS at 37° C. In the absence of laser treatment, bEV(TPP-Ce6) released 56.7% of TPP-Ce6 within 24 h (FIG. 2D). On the other hand, after 1 min of laser irradiation, the cumulative release of TPP-Ce6 reached 86.3% 24 h after the incubation. This result demonstrates that light irradiation effectively accelerated the release of TPP-Ce6 from bEV(TPP-Ce6). The light-triggered release of TPP-Ce6 might be attributed to the destabilization of the EV membranes by light-induced ROS generation from TPP-Ce6. It has been reported that ROS generated from photosensitizers induce lipid peroxidation, which consequently disrupts EVs.

6. Efficient BBB Transcytosis of TPP-Ce6-Loaded bEVs

Penetrating the BBB and delivering therapeutics to brain tumors at therapeutic levels is a significant obstacle to the successful treatment of brain tumors. We hypothesized that brain endothelial cell-derived EVs would cross the BBB through receptor-mediated transcytosis because EVs are known to possess cell-specific proteins found in the membrane of the parent cells. To investigate whether bEVs can penetrate the BBB and enter brain tumor cells, an in vitro BBB model using the transwell method was established. The simulated BBB double-layer was prepared by culturing bEnd.3 cells in the upper chamber and U87MG human GBM cells in the lower chamber. The upper and lower chambers were separated by a porous membrane (FIG. 2E). After bEnd.3 cells in the upper chamber reached a confluent monolayer, they were treated with TPP-Ce6 and bEV(TPP-Ce6) for 4 h, and the amount of TPP-Ce6 released into the lower compartment was quantified. As illustrated in FIG. 2F, encapsulation of TPP-Ce6 into EVs was associated with a significant increase in transport across the BBB, which was indicated by the increased apparent permeability coefficient (Papp) value.

To verify that bEVs cross the BBB, Papp values of rhodamine B (RB)-labeled blank bEVs and RB-labeled bEV(TPP-Ce6) were measured. As shown in FIG. 8, both blank bEVs and bEV(TPP-Ce6) exhibited high Papp values in BBB penetration, demonstrating effective BBB transport of bEVs themselves.

To evaluate the capability of bEV(TPP-Ce6) to cross the BBB and target brain tumors, intracellular uptake levels of Ce6, TPP-Ce6, and TPP-Ce6 by U87MG cells in lower chambers were quantified. The bEnd.3 cells in the upper chambers were treated with Ce6, TPP-Ce6, and bEV(TPP-Ce6) for 4 h. The samples that crossed the bEnd.3 layer could be internalized by U87MG cells in the lower chamber. As presented in FIG. 2G, TPP-Ce6 was more efficiently internalized by the U87MG cells than Ce6. Moreover, bEV(TPP-Ce6) significantly improved the intracellular level of uptake of TPP-Ce6 into U87MG cells. This result indicates that the encapsulation of TPP-Ce6 into bEVs greatly enhances its cellular internalization into GBM. The efficient cellular uptake of bEV(TPP-Ce6) might be attributed to the favorable binding of bEVs with the cell membranes via receptor-ligand interaction or direct fusion.

7. Transferrin-Mediated Brain Targeting of bEVs

Receptor-mediated transcytosis is one of the main routes for the transport of therapeutics through endothelial cells of the BBB. This strategy has been adopted for various therapeutic molecules, including chemical drugs, antibodies, polymeric nanoparticles, and exosomes. The transferrin receptor (TfR), which is overexpressed on the surface of glioma and brain endothelial cells, is a widely validated receptor for the BBB penetration of therapeutics through receptor-mediated transcytosis. Recent studies have reported that blood-derived exosomes express TfR abundantly and exhibit natural BBB-crossing capability through the transferrin-TfR interaction. Based on the efficient BBB penetration capability of bEV(TPP-Ce6), we hypothesized that bEVs could cross the BBB through the transferrin-TfR interaction. Indeed, an enzyme-linked immunosorbent assay verified that bEVs contained high levels of transferrin, which might be bound to TfR expressed on the bEVs (Figure S4). To demonstrate our hypothesis, a transferrin competition binding study was conducted, in which bEnd.3 cells were pretreated with free transferrin prior to the incubation with bEV(TPP-Ce6). Upon pre-incubation with free transferrin, the level of uptake of bEVs by bEnd.3 cells noticeably decreased in a concentration-dependent manner (FIG. 2H). This indicates that pre-treatment with free transferrin saturated TfR on the bEnd.3 cells, leads to a decrease in the amount of available TfR on the surface of bEnd.3 cells. Consequently, cellular internalization of bEV(TPP-Ce6) by bEnd. 3 cells was significantly reduced. This result reveals that the enhanced internalization of the bEV(TPP-Ce6) by bEnd.3 cells is associated with TfR-mediated transcytosis. The same Tf competition binding assay was performed using U87MG cells and hDFB human dermal fibroblast cells. When bEnd.3 cells were replaced with hDFB cells, cellular uptake of bEVs was also reduced by the pre-treatment of Tf (FIG. 2I). However, it should be noted that the pre-treatment of Tf showed a much more significant influence on the cellular uptake of bEVs by bEnd.3 cells than by hDFB cells. The reduction in the cellular uptake of bEVs by U87MG cells was also more significant than that of bEVs by hDFB cells after Tf pre-treatment. This result provides further evidence for the critical role of the transferrin-TfR interaction in the BBB penetration of bEVs. Although our study suggests that bEVs could effectively cross the BBB through the transferrin-TfR interaction, elucidating the exact molecular mechanisms still requires further investigation. In addition to TfR, it has been demonstrated that low-density lipoprotein (LDL) receptors and insulin receptors facilitate the transcytosis of bEVs across the BBB. Further investigation, such as proteomic analysis of bEVs, is needed to elucidate the exact molecular mechanisms underlying the transcytosis of bEVs across the BBB. Moreover, establishing realistic and dynamic in vitro BBB models is crucial to investigate molecular interactions between bEVs and brain endothelial cells.

8. Enhanced Mitochondrial Uptake of Ce6 by Conjugation with TPP

Previous studies have demonstrated that conjugation of TPP to photosensitizers improved their mitochondrial targeting in cells. To demonstrate whether TPP conjugation enhances the mitochondrial uptake of Ce6, mitochondrial uptake levels of Ce6 and TPP-Ce6 in U87MG cells were compared. As shown in FIG. 3A, TPP-Ce6 showed considerably higher mitochondrial uptake in U87MG cells than Ce6, demonstrating that positively charged TPP has high affinity to mitochondrial membranes. Additionally, bEV(TPP-Ce6) showed significantly higher mitochondrial uptake than TPP-Ce6 owing to the increased cellular uptake of bEV(TPP-Ce6).

To demonstrate the mitochondria-targeted localization of TPP-Ce6 in U87MG cells, we performed confocal laser scanning microscopy (CLSM) after incubating U87MG cells with Ce6 and TPP-Ce6. Mander's overlap coefficients were measured to quantitatively determine the colocalization degree of TPP-Ce6 (or Ce6) in the mitochondria, which were labeled with MitoTracker green. The mean fluorescence intensity of U87MG cells treated with TPP-Ce6 was stronger than that of U87MG cells treated with Ce6, indicating higher cellular uptake of TPP-Ce6 than that of Ce6 (FIG. 3B). This result corresponds to the cellular uptake result illustrated in FIG. 2G. Red fluorescence, representing Ce6 or TPP-Ce6, effectively overlapped with the green fluorescence of MitoTracker Green, which was evidenced by a clear yellow fluorescence. Notably, a quantitative colocalization analysis (calculation of Mander's overlap coefficients) demonstrated that TPP-Ce6 was colocalized more efficiently in the mitochondria than Ce6 (e.g., Mander's overlap coefficient of 0.50±0.02 for TPP-Ce6, Mander's overlap coefficient of 0.31±0.03 for Ce6). This result clearly indicates that TPP-Ce6 efficiently targeted the mitochondria of brain tumor cells due to the mitochondria-targeting features of TPP.

9. Cytoplasmic Localization of bEV(TPP-Ce6)

To investigate the intracellular distribution of bEV(TPP-Ce6) in brain tumor cells, rhodamine B (RB)-labeled bEVs (RB-bEVs) were prepared to load TPP-Ce6. The prepared RB-labeled bEV(TPP-Ce6) was added to bEnd.3 and U87MG cells. Both bEnd.3 and U87MG cells were stained with LysoTracker, which preferentially stains endo/lysosomal compartments, to investigate whether bEV(TPP-Ce6) can enter cells via the endocytic pathway. As illustrated in FIG. 11, a large amount of RB-bEVs (red) were colocalized with LysoTracker (green) in the cells. This result verifies that bEV(TPP-Ce6) enters cells via endocytosis. Intracellular distributions of RB-bEVs (red dots) and TPP-Ce6 (cyan dots) in U87MG cells after incubation with RB-labeled bEV(TPP-Ce6) are shown in FIG. 3C. Both RB-bEVs and TPP-Ce6 were distributed primarily in the cytoplasm. Moreover, significant amounts of RB-bEV and TPP-Ce6 were localized in the mitochondria (green) stained with MitoTracker Green. The Mander's overlap coefficient values of mitochondria with TPP-Ce6 were 2 times higher than those with RB-bEVs in U87MG cells, indicating that the TPP-Ce6 released from bEVs efficiently accumulated in the mitochondria.

10. Light-Triggered Intracellular ROS Generation Using TPP-Ce6-Loaded bEVs

The efficacy in ROS generation of photosensitizers under light illumination is critical for photodynamic cancer therapy. To investigate whether TPP-Ce6 produces ROS upon 660-nm light irradiation, the relative ROS production from free Ce6 solution, free TPP-Ce6 solution, and bEV(TPP-Ce6) suspensions in a cell-free system was quantified before and after 660-nm laser treatment. The laser irradiation significantly increased the ROS generation of Ce6 and TPP-Ce6 (FIG. 12). Notably, bEV(TPP-Ce6) exhibited a superior capability to produce ROS compared to TPP-Ce6. This finding might be attributed to the increased stability of TPP-Ce6 in aqueous solutions when encapsulated in bEVs (FIG. 2C). These results demonstrate that bEV(TPP-Ce6) is an effective photosensitizer for PDT.

Conjugation of TPP facilitates the intracellular transport of photosensitizers to mitochondria and thus affects their intracellular ROS generation. To examine whether the conjugation of TPP to Ce6 affects the intracellular ROS generation of Ce6, intracellular ROS levels in U87MG cells were treated with Ce6, TPP-Ce6, and bEV(TPP-Ce6) before and after light irradiation. TPP-Ce6 significantly increased the intracellular ROS generation in U87MG cells relative to Ce6, regardless of light irradiation (FIG. 3D). The encapsulation of TPP-Ce6 in bEVs drastically elevated intracellular ROS generation in U87MG cells. This result can be explained by the enhanced cellular internalization of bEV(TPP-Ce6) compared to TPP-Ce6. Notably, the intracellular ROS levels in U87MG cells after treatment with Ce6, TPP-Ce6, and bEV(TPP-Ce6) were significantly increased after 1 min of light exposure (FIG. 3D). This result demonstrated the efficient photodynamic effects of Ce6. Fluorescence images of 2′,7′-dichlorofluorescein diacetate (DCF-DA)-stained U87MG cells were obtained to observe light-triggered ROS generation by Ce6, TPP-Ce6, and bEV(TPP-Ce6). As displayed in FIG. 3E, we observed weak fluorescence signals in the cells treated with Ce6. The fluorescence signals increased when the cells were treated with TPP-Ce6 or bEV(TPP-Ce6). The fluorescence signals in the cells increased significantly when the cells were exposed to a 660-nm laser. The highest fluorescence signals were observed within the cells that received bEV(TPP-Ce6) and light irradiation, indicating the high efficacy of bEV(TPP-Ce6) in ROS generation. This might be ascribed to the efficient cellular internalization of bEV(TPP-Ce6).

11. In Vitro PDT Using Mitochondria-Targeting TPP-Ce6-Loaded bEVs

Mitochondria are key organelles of cellular energy metabolism. When mitochondria are damaged, they initiate the loss of mitochondrial membrane potential and cascades for apoptosis. Therefore, mitochondria are attractive targets for effective PDT. A conventional JC-1 staining assay was used to determine whether mitochondria were damaged after various treatments. In healthy cells with high mitochondria membrane potential, the JC-1 dye accumulates in the mitochondria and forms JC-1 aggregates, emitting red fluorescence. However, in apoptotic cells with reduced mitochondria membrane potential, JC-1 dye exists as a monomer emitting green fluorescence. Consequently, mitochondrial damage was indicated by a decrease in the red/green fluorescence intensity ratio. As displayed in FIG. 4A, strong red fluorescence was observed in the control group, indicating a minor change in mitochondrial membrane potential. The red fluorescence in the cells drastically decreased when they were treated with Ce6 and TPP-Ce6 under light irradiation. Importantly, the cells treated with bEV(TPP-Ce6) plus laser irradiation showed the lowest fluorescence ratio of red to green, indicating the most severe mitochondrial damage. This result demonstrates that mitochondria-targeting PDT by bEV(TPP-Ce6) effectively damaged the mitochondria.

The viability of U87MG cells after various treatments under laser irradiation was analyzed using a standard MTT assay. As shown in FIG. 4B, more than 92% of the cells retained viability upon 1 min of light irradiation only, suggesting that light irradiation is minimally cytotoxic. The viability of U87MG cells slightly decreased when they were treated with Ce6 and TPP-Ce6. The cytotoxicity of bEV(TPP-Ce6) was substantially higher than that of free TPP-Ce6. The increased uptake of TPP-Ce6 by bEVs might have contributed to this increased cytotoxicity. A significant drop in cell viability was found when the cells received Ce6 or TPP-Ce6 upon exposure to light. The viabilities of the cells that received Ce6 and TPP-Ce6 under light irradiation were 72.5 and 42.2%, respectively (FIG. 4B). The higher phototoxicity of TPP-Ce6 compared to Ce6 might be attributed to the increased mitochondrial damage that leads to apoptosis. Notably high phototoxicity (8.9% cell viability) was observed when bEV(TPP-Ce6) was added to the cells under light irradiation (FIG. 4B). This is attributed to the efficient cellular uptake and mitochondrial targeting of bEV(TPP-Ce6). The photodynamic effects of various samples were observed by a live/dead staining assay. Fluorescence images of the dye-stained U87MG cells that received various treatments are provided in FIG. 4C. The treatment with bEV(TPP-Ce6) plus laser irradiation showed the highest percentage of dead cells, which is consistent with the cell viability results (FIG. 4B).

The cytotoxicity of the blank bEVs against U87MG cells was assessed to demonstrate their biosafety. The blank bEVs exhibited very minimal cytotoxicity (>90% viability) up to a concentration of 1.0×10¹¹ bEVs/mL (FIG. 4D). The bEV and TPP-Ce6 concentrations used for the in vitro cytotoxicity study (FIG. 4A) were approximately 5.38×10¹⁰ bEVs/mL and 10 μM, respectively. The intrinsic toxicity of drug carriers remains a major obstacle that makes it challenging to use them in a clinical states. Therefore, the low toxicity of bEVs suggests that bEV-based photosensitizers have great promise in facilitating the clinical translation of brain tumor-targeted PDT.

To investigate whether phototoxicities of TPP-Ce6 and bEV(TPP-Ce6) result from apoptosis, apoptosis rates of U87MG cells treated with TPP-Ce6 and bEV(TPP-Ce6) were analyzed before and after 660 nm light irradiation (FIG. 4E). An annexin V-fluorescein isothiocyanate (FITC)/PI staining assay revealed that U87MG cells treated with bEV(TPP-Ce6) showed a higher level of total apoptosis than those treated with TPP-Ce6, regardless of laser exposure. A dramatic increase in the population of total apoptotic cells was found when the cells received TPP-Ce6 under light irradiation. Treatment with bEV(TPP-Ce6) combined with light irradiation significantly increased the percentage of total apoptotic (early and late apoptosis) cells from 28.14 to 66.80%. The apoptosis results corresponded well to the cytotoxicity results (FIG. 4B). Together with the intracellular ROS generation analysis (FIG. 3D), the apoptosis results demonstrate that elevated oxidative stress from the photodynamic effects of bEV(TPP-Ce6) triggers apoptosis in GBM cells.

12. Effective Brain Tumor Accumulation of bEV(TPP-Ce6)

In vivo biodistribution and brain tumor accumulation of Ce6, TPP-C e6, and bEV(TPP-Ce6) were evaluated using luciferase-expressing U87MG GBM-xenograf ted mice. The samples were intravenously administered to the orthotopic GBM-xenograft ed mice via the tail vein. The distribution of Ce6, TPP-Ce6, and bEV(TPP-Ce 6) in the body was determined by measuring the fluorescence from Ce6 and TPP-Ce6 using IVIS (FIG. 5A). Within 10 min after intravenous (i.v.) injection, the fluor escence signals of Ce6, TPP-Ce6, and bEV(TPP-Ce6) were detected across the whole bo dy—that is, the fluorescence signal of bEV(TPP-Ce6) circulating in the blood. While weak fluorescence signals were observed in the brains of mice treated with Ce6 and TP P-Ce6 at 24 h post-injection, the bEV(TPP-Ce6)-treated mice showed high fluorescence in tensity in the brain at 24 h after injection.

The time courses of the whole brain-to-whole body fluorescence intensity ratios at 1 min post-injection were also measured to quantitatively estimate the brain-selective accumulation of Ce6, TPP-Ce6, and bEV(TPP-Ce6). The time courses of the whole brain-to-whole body fluorescence ratios for Ce6- and TPP-Ce6-treated mice were similar (FIG. 5B). In contrast, bEV(TPP-Ce6)-treated mice exhibited higher whole brain-to-whole body fluorescence ratios than Ce6- and TPP-Ce6-treated mice. These results suggest that bEV(TPP-Ce6) could effectively transport TPP-Ce6 into the brain across the BBB. To evaluate the GBM tumor-targeting capability of Ce6, TPP-Ce6, and bEV(TPP-Ce6) in mice, time course brain tumor-to-whole brain fluorescence ratios were measured after i.v. injection of Ce6, TPP-Ce6, and bEV(TPP-Ce6). As shown in FIG. 5C, bEV(TPP-Ce6)-treated mice displayed higher brain tumor-to-whole brain fluorescence ratios over time compared to Ce6- and TPP-Ce6-treated mice. These results demonstrate that bEVs can significantly enhance the accumulation of TPP-Ce6 in brain tissue and brain tumors. It should be noted that the brain tumor-to-whole brain fluorescence ratio was found to be highest at 4 h post-injection of bEV(TPP-Ce6). Therefore, the mice were irradiated with light 4 h after the administration of bEV(TPP-Ce6) to achieve the most effective PDT using bEV(TPP-Ce6).

Ex vivo imaging of the GBM-bearing brains and major organs collected 24 h after injection was performed. All the mice showed the highest level of fluorescence signal in the liver at 24 h post-injection (FIG. 5D). Notably, at 24 h post-administration, the level of TPP-Ce6 localization in the brain of the bEV(TPP-Ce6)-treated mice was significantly higher than that for TPP-Ce6-treated mice, which corresponded to the real-time biodistribution result (FIG. 5A).

In situ accumulation of bEV(TPP-Ce6) in brain tumors of mice after i.v. injection was evaluated using CLSM. To observe the brain tumor accumulation of bEV(TPP-Ce6), FITC was used to label bEVs. bEV(TPP-Ce6) labeled with FITC was intravenously injected into orthotopic GBM-xenografted mice. Brain tissue was collected 2 h after the injection, and localization of FITC-labeled bEV(TPP-Ce6) around the boundary between normal and tumor tissue in brain slices was assessed using CLSM. As shown in FIGS. 5F and 5G, much stronger green fluorescence [FITC-labeled bEV (FITC-bEV)] and red fluorescence (TPP-Ce6) were detected at the tumor site compared to the normal site. This result indicates that bEV(TPP-Ce6) accumulates preferentially in brain tumor tissue.

13. Efficient In Vivo PDT Effects of bEV(TPP-Ce6)

The in vivo PDT effects of bEV(TPP-Ce6) were evaluated using luciferase-expressing U87MG-bearing mice. Representative images of bioluminescence intensity (BLI) in brain tumors of the mice treated with PBS, Ce6, TPP-Ce6, and bEV(TPP-Ce6) are displayed in FIG. 6A. The mice were illuminated with a 660 nm laser for 5 min at 4 h post i.v. injection of each sample. The BLI in the brain tumors treated with PBS increased dramatically for 9 days, regardless of light irradiation (FIG. 6A). Free Ce6- and TPP-Ce6-treated mice showed limited suppression of tumor growth upon laser treatment. In contrast, treatment with bEV(TPP-Ce6) plus laser irradiation showed the most significant suppression of tumor growth (FIGS. 6A and 6B). Safety has been a major concern in relation to the use of synthetic nanocarriers for PDT. Notably, all the samples caused negligible body weight changes over 9 days, indicating that no adverse effects were induced by the samples (FIG. 6C).

The efficient PDT effects of bEV(TPP-Ce6) were also confirmed by H&E staining of brain sections from GBM-xenografted mice. As displayed in FIG. 6D, the brain tumor tissue of the mice injected with PBS exhibited extremely rich and closely arranged tumor cells, regardless of light irradiation. In contrast, the tumor tissue of the mice treated with free Ce6, TPP-Ce6, and bEV(TPP-Ce6) upon light irradiation showed low cell density and nuclear shrinkage. The smallest tumor size and largest area of necrotic change and nuclear shrinkage in tumor tissue were observed in bEV(TPP-Ce6)-treated mice subjected to light irradiation, indicating the high PDT efficacy of bEV(TPP-Ce6) for the treatment of GBM (FIG. 6D). Negligible pathological changes were observed in the major organs of mice in all groups, demonstrating the high biosafety of bEV(TPP-Ce6) for PDT (FIG. 6E). These results demonstrate that bEV(TPP-Ce6) combined with light irradiation can efficiently inhibit the growth of GBM without causing adverse effects.

The present disclosure may be implemented in various modified manners within the scope of the present disclosure that do not depart from the technical idea of the present disclosure. Accordingly, the intention behind describing the embodiments of the present disclosure is not to limit the technical idea of the present disclosure, but to describe the present disclosure. The scope of the technical idea of the present disclosure is not limited by the embodiments. Therefore, it should be understood that the embodiments as described above are illustrative and non-limiting in all respects. The scope of protection of the present disclosure should be interpreted by the claims, and all technical ideas within the scope of the present disclosure should be interpreted as being included in the present disclosure.

What is claimed is:

1. A drug delivery system for penetrating the blood-brain barrier containing extracellular vesicles (bEVs) derived from brain vascular endothelial cells as active ingredients.

2. A composition for enhancing the efficacy of photodynamic therapy, which includes photosensitizers loaded on extracellular vesicles (bEVs) derived from brain vascular endothelial cells as an active ingredient.

3. The composition of claim 2, wherein the photosensitizer is conjugated with Triphenylphosphonium(TPP).

4. The composition of claim 2, wherein the photosensitizer is chlorin e6(Ce6).

5. The composition of claim 2, wherein the composition is administered 3 to 6 hours before photodynamic therapy is performed.

6. A composition for treating brain tumors that includes photosensitizers loaded on extracellular vesicles (bEVs) derived from brain vascular endothelial cells as an active ingredient.

7. The composition of claim 6, wherein the composition is administered before photodynamic therapy is performed.

8. The composition of claim 6, wherein the composition is administered 3 to 6 hours before photodynamic therapy is performed.

9. The composition of claim 6, wherein the brain tumor is a glioblastoma.

10. The composition of claim 6, wherein the photosensitizer is conjugated with Triphenylphosphonium(TPP).

11. The composition of claim 6, wherein the photosensitizer is chlorin e6(Ce6).
